(19) United States
(12) Patent Application Publication
(10) Pub. No.: US 2009/0131841 A1
(43) Pub. Date: May 21, 2009

(54) Thorax Supporting Device

(75) Inventors: Jurgen Epple, Vienna (AT); Lars Svensson, Gates Mills, OH (US)

Correspondence Address: CHAPMAN AND CUTLER, 111 WEST MONROE STREET, CHICAGO, IL, 60603, US

(73) Assignees: Garvington Research & Development Inc., Clearwater, FL (US); Heartsease Therapy , LLC., Gates Mills, OH (US)

(21) Appl. No.: 12/084,179

(22) PCT Filed: Oct. 27, 2006
(86) PCT No.: PCT/AT2006/000440

§ 371 Date: Nov. 19, 2008

(30) Foreign Application Priority Data
Oct. 28, 2005 (AT) A 1768/2005

(51) Int. Cl. A61F 5/00

(52) U.S. Cl. 602/19

(57) ABSTRACT

In a thorax supporting device including at least one tension element (1) surrounding the thorax and support elements capable of being pressed against the thorax by the aid of the at least one tension element, the support elements are formed by at least two cushions (2, 3) or the like, which are arranged on both sides of the thoracic part to be supported. The tension element (1) comprises at least two portions of different elasticity, wherein a first portion (13) extending between the two cushions (2, 3) over the thoracic region to be supported has a lower elasticity in the tension direction than a second portion (14) extending between the cushions (2, 3) in the opposite direction.

The invention relates to a thorax supporting device including at least one tension element surrounding the thorax and support elements capable of being pressed against the thorax by the aid of the at least one tension element.

Supporting devices of this type are, for instance, used as rib fracture belts and serve to stabilize the thorax after a rib fracture in order to accelerate healing. Such a rib fracture belt is, for instance, formed by a girdle surrounding the thorax, which becomes active as a tension or clamp element to cause a limitation of the thoracic expansion during breathing by appropriate clamping. Such a rib fracture belt will additionally support the spinal column, in particular after a thoracotomy, so as to counteract backaches.

A rib fracture belt is, for instance, known from GB 626425 A.

From WO 96/28129 A1, a similar device has become known, which is, however, employed as a cardiopulmonary resuscitation device rather than a rib breast belt. That device is formed by a thorax-surrounding belt on whose inner side inflatable cushions are provided to exert a suitable pressure on the thorax.

A stabilization of the thorax is not only required after rib breaks, but also after a thoracotomy such as, for instance, in the course of a heart surgery. After having closed the thorax, the respective wound must be stabilized in order to ensure rapid healing, said stabilization being, however, complicated by the thoracic expansion occurring during breathing. In particular, during coughing and other sudden expansions of the thorax, the wound will be subjected to elevated strains, and the present invention, therefore, aims to provide a supporting device of the initially defined kind, which provides sufficient stabilization of the thorax in the region of the wound to accelerate healing yet, at the same time, allows for the thoracic expansion caused by breathing, while saving the wound area.

To solve this object, the supporting device according to the invention is essentially characterized in that the support elements are formed by at least two cushions or the like, which are arranged on both sides of the thoracic part to be supported, and that the tension element comprises at least two portions of different elasticity, wherein a first portion extending between the two cushions over the thoracic region to be supported has a lower elasticity in the tension direction than a second portion extending between the cushions in the opposite direction. Due to the fact that the support elements are formed by at least two cushions or the like, which are arranged on both sides of the thoracic part to be supported, it will, on the one hand, be prevented that the tension or clamp element rests directly on the thoracic part to be supported and, in particular, on the wound formed by the thoracotomy and, on the other hand, be ensured that the thoracic region located between the cushions is substantially kept free from expansions. To this end, it is, in fact, further provided according to the invention that the tension element has a lower elasticity in its portion extending between the two cushions over the thoracic region to be supported than the remaining portion of the tension element. The low elasticity of the tension element in its portion extending over the thoracic region to be supported, or the fact that the tension element preferably has substantially no elasticity at all in said portion, causes the distance between the two cushions delimiting the wound to be kept substantially constant such that no thoracic expansion will come into effect in this sensitive region. The thoracic expansion rather will come into effect only in the portion extending between the cushions in the opposite direction and, hence, only in the region in which the thorax is intact and, thus, does not require any wound healing. The operation seam of the thoracotomy will thereby be protected in the post-operative area, i.e., for instance, after a heart or lung operation, and the risk of a bursting of the seam will be counteracted, thus alleviating postoperative pain.

In order to adapt the supporting device according to the invention to the respective requirements, the configuration is preferably further developed such that the length of the first portion of the tension element is adjustable. The effective length of the tension or clamp element acting as a spacer between the two cushions in the region of the thoracic part to be supported can, thus be adjusted while, at the same time, adapting the overall pressure exerted on the thorax.

In order to safeguard a constant distance between the two cushions, it must, of course, be taken care that the cushions keep their positions relative to the thorax without slipping, because slipping of the cushions would immediately cause strain and, hence, a destabilization of the wound area. In this context, it has to be ensured, on the one hand, that the cushions are made to be appropriately slip-free, and it is, therefore, provided according to a preferred further development of the supporting device according to the invention that the cushions are each provided with a friction-enhancing layer on their sides facing the thorax. On the other hand, a sufficiently high pressure of the cushions against the thorax must be ensured in the radial direction, which, in the design according to the invention, is already promoted in that the tension or clamp element surrounding the thorax will act on the thorax via the interposed cushion. The tension or clamp element in the region to be supported will, thus, not directly rest on the thorax, and the force caused by the tension or clamp element in the peripheral direction will thereby be effectively decomposed into a radially acting component in the region of the cushions. The higher the cushion, i.e. the larger the distance of the tension element from the thorax in the region of the cushion, the higher the force exerted on the thorax by the cushion.

As already mentioned above, the thoracic expansion occurring during breathing is allowed by the supporting device according to the invention to the extent permitted by the elasticity of the tension or clamp element, said elasticity being primarily provided in that region of the tension or clamp element, which is located outside the thoracic region to be supported. The second portion of the tension or clamp element, however, need not be elastically designed over the entire length, and it is preferably provided that the second portion comprises expansion zones limited in elasticity and has substantially no elasticity outside said expansion zones. Such a configuration renders feasible the limitation and control of the permissible expansion of the tension or clamp element, since any further expansion will be prevented if the expansion permitted by the expansion zones is exceeded. In this manner, the supporting effect will be improved and the stability of the supporting device will be enhanced.

In order to adapt the supporting device to the respective thoracic circumference, the configuration is preferably further devised such that the length of the second portion is adjustable.

In a preferred manner, the tension element is comprised of at least two straps or belts each surrounding the thorax in a mutually spaced-apart relationship. By providing a plurality of straps or belts, an improved adaptation to the respective shape of the body is feasible, and different length adjustments can, for instance, be adopted for the individual straps or belts. Also the widths of the individual straps or belts can be chosen to differ from one another in order to achieve an optimum pressure distribution and, hence, an improved wearing comfort. A wider strap may, for instance, be used if the strap in question has to exert higher tensile forces in order to reach a uniform surface pressure as compared to other straps. The free space remaining between the individual straps or belts serves to enhance air circulation, thus reducing local heating of the skin.

In order to further enhance the wearing comfort, it may preferably be provided that the second portion of the tension or clamp element carries additional cushions which come to lie between the tension element and the thorax, the arrangement of the additional cushions being preferably devised such that said additional cushions come to lie in the side region of the thorax. As a result, a pressure distribution over a larger surface will be reached, and constrictions of the skin or possibly present adipose tissue by individual belts will, therefore, be prevented. Additional cushions may, however, also be provided in the dorsal region in order to increase the comfort level.

In order to enhance the variability of the supporting device, it is preferably provided that the cushions are detachably connected with the tension element. The detachable connection may be achieved in a conventional manner, for instance by the aid of hook-and-loop fasteners, and ensures easy exchangeability, for instance for cleaning purposes. Moreover, the option to choose among different cushioning means will be provided.

In order to improve the stability of the supporting device, it may preferably be provided that the tension element is connected with two shoulder straps. Such shoulder straps will prevent the supporting device from sliding off the patient's thorax. Clamping of the shoulder straps will, moreover, cause a simultaneous increase of the tension in the peripheral direction, i.e. an increase in the pressure on the thorax.

Finally, the tension element and/or the back part may be provided with pockets for receiving therapeutic aids such as, e.g. magnets or electrically stimulating electrodes. However, such pockets may, for instance, also be used for cooling elements, if this is desired in individual cases, for instance for the relief of pain.

In the following, the invention will be explained in more detail by way of an exemplary embodiment schematically illustrated in the drawing.

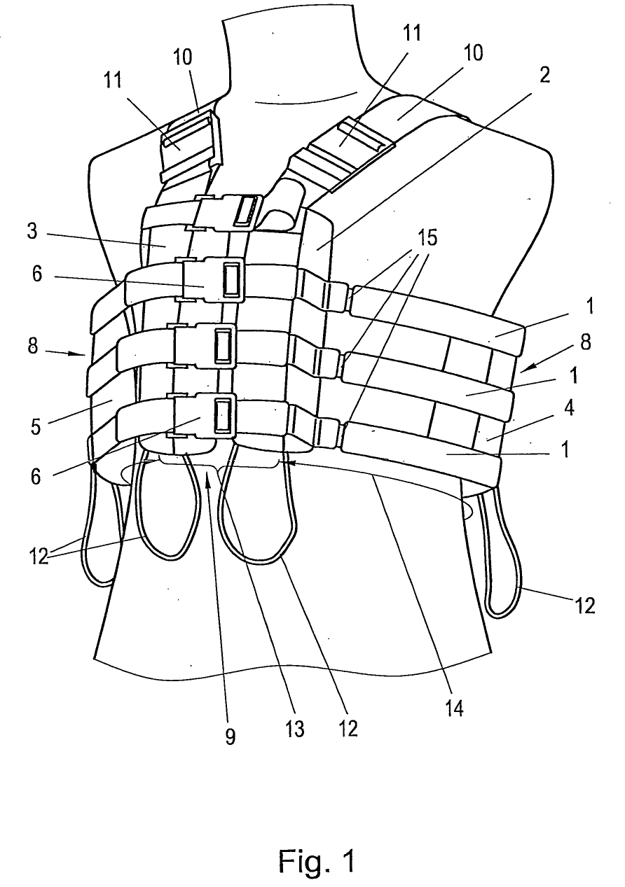

Therein, FIG. 1 is a front view of a supporting device according to the invention applied on a dummy;

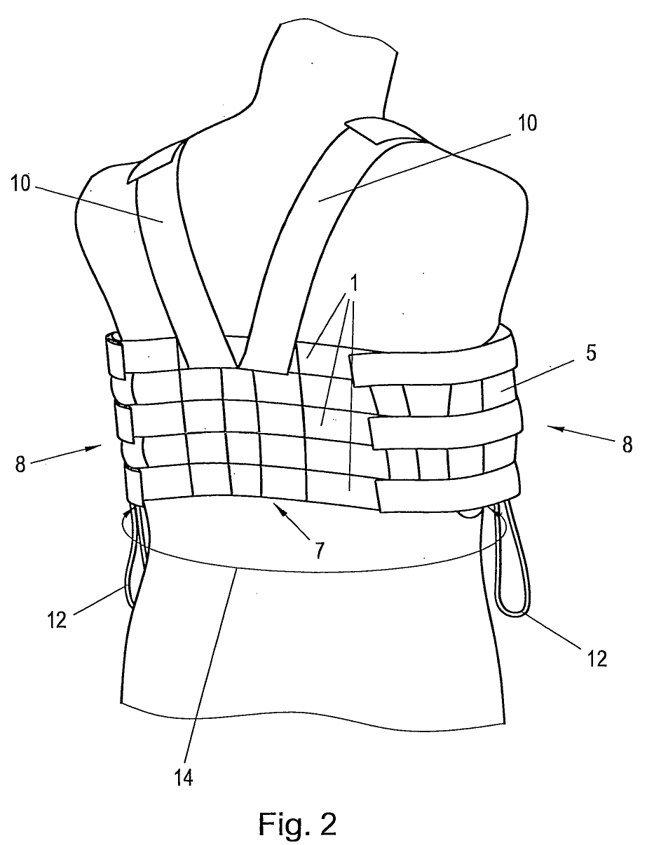

FIG. 2 is a back view; and

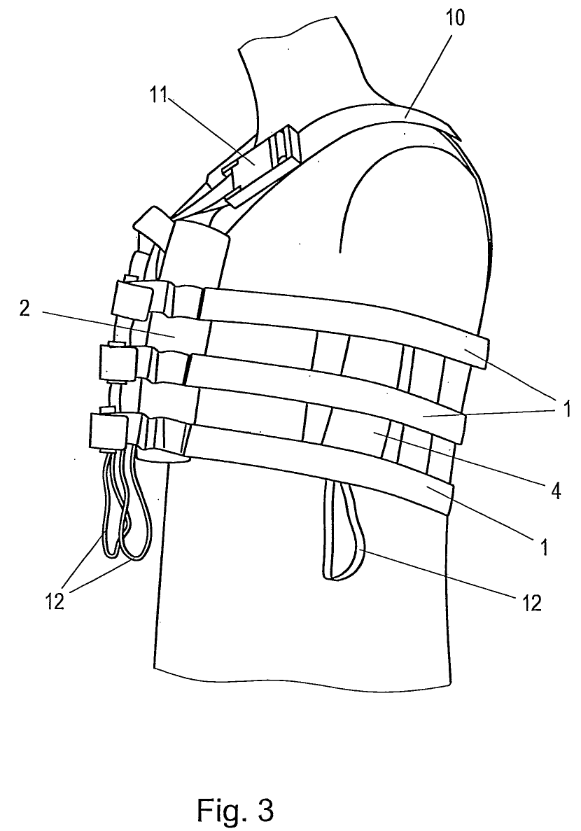

FIG. 3 is a side view.

From the Figures, it is apparent that the supporting device according to the invention essentially consists of a tension or clamp element surrounding the thorax as well as cushions which are pressed against the thorax by the tension element. The tension or clamp element in the present case is comprised of three straps (tension elements) 1 each encompassing the thorax in a mutually spaced-apart relationship and being connected with, or engaging, cushions 2, 3, 4 and 5, respectively. Between the cushions 2 and 3 is located the thoracic region to be supported, i.e., in the present case, the region around the sternum, where the thorax was incised to perform a heart or lung surgery. The cushions 2 and 3 may be made of a conventional material and are connected with the straps (tension elements) 1, or front part 9, for instance, by the aid of a hook-and-loop fastener to ensure easy exchangeability. On the cushion side facing the thorax, a friction-enhancing layer made, for instance, of Neopren may be provided to prevent the cushions from slipping on the skin. The cushions 2 and 3 may also be connected to form a single cushion element. Such a cushion element will rest on the thorax on both sides of the thoracic region to be supported and will include a recess in the thoracic region to be supported, thus having, for instance, U-shaped cross section.

The tension elements 1 surrounding the thorax may each be formed in one piece in the peripheral direction, wherein buckles 6 are provided in their central regions to facilitate putting on and off. In the present case, no one-piece configuration of the tension straps 1 is, however, provided, but a back part 7 is arranged, which is connected with a side part 8 on either side, each by the aid of, for instance, hook-and-loop fastening means. The side parts 8, in turn, extend as far as to the cushions 2 and 3, respectively, said two side parts 8 being connected with each other by the front part 9. Between the front part 9 and the back part 7, two shoulder straps 10 are provided, which can again be opened by the aid of buckles 11 and are adjustable in length. The straps 10 prevent the supporting device from slipping down. However, should the supporting device slip upwards, loops 12 are provided in front and/or laterally, by which the supporting device can be manually pulled down.

The tension element formed by the tension straps 1 comprises a first portion 13 extending between the two cushions 2 and 3. In said first portion 13, the tension straps 1 are substantially inelastic such that a constant distance will be maintained between the cushions 2 and 3. Provided the cushions 2 and 3 do not slip relative to the thorax, this will cause the stabilization of the thoracic region located between the cushions 2 and 3 and of the wound caused by the thoracotomy. The risk of destabilization will, in fact, exist during the thoracic expansion caused by breathing and, in particular, at sudden thoracic expansions such as, for instance, during coughing. The two cushions 2 and 3, which are constantly held at the same distance from each other, will effectively prevent a thoracic expansion in the wound area, and an expansion of the thorax will rather occur merely in the remaining region of the thorax outside the area limited by the two cushions 2 and 3. In this second portion 14 of the tensions straps 1, i.e. in the remaining peripheral portion extending in the opposite direction between the cushions 2 and 3, the expansion of the thorax will cause a change in the length of the tension straps 1 within the range of elasticity of the same. To this end, the tension straps 1 include expansion zones 15 which are limited in elasticity. The expansion zones 15 are hereby comprised of an elastic strap portion as well as an expansion-free strap portion parallel thereto, which will only enter into action after a pregiven expansion of the elastic portion has been exceeded, thus preventing any further expansion.

In the present exemplary embodiment, only square-shaped cushions are shown. Yet, the cushions may, of course, also be oblique or of any other shape and have different dimensions in terms of length, width and thickness. Cooling elements may also be integrated in the cushions.

In order to render the application also comfortable for women, the central one of the three tension elements 1 may be omitted and, for instance, replaced with a brassiere that is integrated in the supporting device. The material used for the tension straps 1 may be comprised of a fabric or a synthetic material.

1. A thorax supporting device, comprising:
at least one tension element that surrounds the thorax; and
support elements adapted to being pressed against the thorax by pressure from the at least one tension element, wherein
the support elements are at least two cushions, which are arranged on both sides of a thoracic region to be supported, and
the tension element comprises at least two portions having different elasticity, with
a first one of said portions that extends between the cushions over the thoracic region to be supported having a lower elasticity in the tension direction than the elasticity of a second one of said portions that extends between the cushions in an opposite direction that is not over the thoracic region to be supported.

2. A supporting device according to claim 1, wherein the first portion of the tension element, which extends between the cushions over the thoracic region to be supported, has no elasticity in the tension direction.

3. A supporting device according to claim 1, wherein a length of the first portion is adjustable.

4. A supporting device according to claim 1, wherein the second portion comprises expansion zones limited in elasticity, and said second portion has substantially no elasticity outside said expansion zones.

5. A supporting device according to claim 1, wherein a length of the second portion is adjustable.

6. A supporting device according to claim 1, wherein the cushions are each provided with a friction-enhancing layer on a side of the cushion facing the thorax.

7. A supporting device according to claim 1, wherein the tension element is comprised of at least two tension straps each surrounding the thorax, said straps being arranged in a mutually spaced-apart relationship.

8. A supporting device according to claim 1, wherein the second portion of the tension element carries additional cushions that lie on one or more of side regions and a back region of the thorax.

9. A supporting device according to claim 1, wherein the cushions are detachably connected with the tension element.

10. A supporting device according to claim 1, wherein the tension element is connected with two shoulder straps.

11. A supporting device according to claim 1, wherein the tension element comprises pockets for receiving therapeutic aids.

12. A supporting device according to claim 2, wherein a length of the first portion is adjustable.

13. A supporting device according to claim 2, wherein the second portion comprises expansion zones limited in elasticity, and said second portion has substantially no elasticity outside said expansion zones.

14. A supporting device according to claim 3, wherein the second portion comprises expansion zones limited in elasticity, and said second portion has substantially no elasticity outside said expansion zones.

15. A supporting device according to claim 2, wherein a length of the second portion is adjustable.

16. A supporting device according to claim 3, wherein a length of the second portion is adjustable.

17. A supporting device according to claim 4, wherein a length of the second portion is adjustable.

18. A supporting device according to claim 2, wherein the cushions are each provided with a friction-enhancing layer on a side of the cushion facing the thorax.

19. A supporting device according to claim 3, wherein the cushions are each provided with a friction-enhancing layer on a side of the cushion facing the thorax.

20. A supporting device according to claim 4, wherein the cushions are each provided with a friction-enhancing layer on a side of the cushion facing the thorax.
